(19) United States
(12) Patent Application Publication
(10) Pub. No.: US 2006/0105392 A1
(43) Pub. Date: May 18, 2006

(54) Differential diagnosis of vitamin B12, vitamin B6, and folic acid disorders

(76) Inventors: Paul Lehmann, Worms (DE); Ralf Roddiger, Gorxheimertal (DE); Horst Klima, Penzberg (DE)

Correspondence Address: Roche Diagnostics Corporation, 9115 Hague Road, PO Box 50457, Indianapolis, IN, 46250-0457, US

(21) Appl. No.: 11/225,287

(22) Filed: Sep. 13, 2005

Related U.S. Application Data
(63) Continuation of application No. PCT/EP04/02539, filed on Mar. 11, 2004.

(30) Foreign Application Priority Data
Mar. 13, 2003 (DE) DE 10311089.5

(51) Int. Cl. G01N 33/53; G01N 33/00

(52) U.S. Cl. 435/7.1; 436/811; 436/90

(57) ABSTRACT

The invention concerns a method for determining vitamin B12, B6 or/and folic acid disorders and in particular the differential diagnosis of vitamin B12, vitamin B6 or/and folic acid disorders by means of three or four independent parameters. The differential diagnosis can be used to detect a vitamin B12, vitamin B6 or/and folic acid disorder and to recommend the required treatment and to monitor the course and success of treatment.

### RELATED APPLICATIONS

This application is a continuation of international application PCT/EP2004/002539 filed Mar. 11, 2004, and claims priority to German application DE 10311089.5 filed Mar. 13, 2003.

### FIELD OF THE INVENTION

The invention concerns a method for determining vitamin B12, vitamin B6 and/or folic acid disorders and in particular the differential diagnosis of vitamin B12, vitamin B6 and/or folic acid disorders by means of three or four independent parameters. The differential diagnosis can be used to detect a vitamin B12, vitamin B6 and/or folic acid deficiency and to recommend the required treatment and to monitor the course and success of treatment.

### BACKGROUND

Vitamins B12, B6 and folic acid are of major importance in the human organism as precursor substances for the formation of coenzymes. B12 and folic acid deficiencies occur very frequently and can produce various deficiency symptoms and diseases, and they are also a risk factor for numerous diseases. Thus, for example, non-inflammatory chronic diseases are often characterized by a deficiency of B vitamins (B12, B6, folic acid).

In the human organism vitamin B12 is absorbed in the gastric mucosa by binding to the so-called intrinsic factor which specifically binds vitamin B12. Vitamin B12 reaches the ileum in its bound form where it is taken up into the epithelium by endocytosis. Vitamin B12 is cleaved by the intrinsic factor and bound to transcobalamin II inside the mucosal cells of the ileum. The complex of transcobalamin II and vitamin B12 (holotranscobalamin II, holo-TC II) leaves the cell and can be distributed within the organism. Large amounts of vitamin B12 are stored in the liver (ca. 4 to 5 g).

As a coenzyme, vitamin B12, partly together with folic acid, plays an essential role in fat, carbohydrate and nucleic acid metabolism. Among other things, vitamin B12 is indispensable for normal erythropoiesis and nerve cell function. The metabolism of vitamin B12 is closely linked to that of folic acid. In their active form, both vitamins are involved in C1 metabolism as coenzymes.

Tetrahydrofolic acid, the form of folic acid which is active as a coenzyme, plays a major role in the transfer of C1 units and thus, for example, influences nucleic acid synthesis, amino acid metabolism and the formation of blood cells.

Vitamin B12 deficiency can, for example, be caused by malnutrition, by malabsorption or by defects in the absorption or transport mechanisms for vitamin B12. However, serious deficiency symptoms are only likely to occur after several years since the body has a very high storage capacity for vitamin B12.

According to Herbert (Am. J. Clin. Nutr. 1994; 59 (suppl.): 1213S-22S) the transition from a normal vitamin B12 status to vitamin B12 deficiency can be subdivided into four stages. The first stage is usually characterized by a reduced vitamin B12 concentration in the serum. In the second stage it is already possible to observe a depletion and the onset of a reduction in the store of vitamin B12 in the cells, and in the third stage there is already a biochemical vitamin B12 deficiency with severe functional disorders such as defective erythropoiesis. The fourth stage is a clinically manifest vitamin B12 deficiency in which anaemia and nerve damage may be present. Depending on the duration of the deficiency state, damage may occur that is no longer reversible.

Vitamin B12 deficiency in humans leads to pernicious anaemia which is a form of megaloblastic anaemia. Furthermore funicular myelosis may occur which is a severe degeneration of certain areas of the spinal cord. The haematological symptoms of a vitamin B12 deficiency are similar to those of a folic acid deficiency.

Folic acid deficiency is the most widespread vitamin deficiency after vitamin B12 deficiency. Folic acid deficiency may for example be due to malnutrition, malabsorption, an increased requirement e.g. during pregnancy or lactation, an increased elimination e.g. during long-term haemodialysis or due to drug-induced disorders.

Tetrahydrofolic acid for example plays a key role as a coenzyme in thymidylate synthesis. Since vitamin B12 is also involved as a coenzyme in this synthesis, a vitamin B12 deficiency may also result in a functional folic acid deficiency.

The possibilities for storing folic acid are limited in the human body. The folic acid stores of the liver are sufficient to maintain a normal folic acid level in the serum for only about three to four weeks.

Folic acid deficiency leads to megaloblastic anaemia in humans. However, as a result of the close linkage between folic acid metabolism and vitamin B12 metabolism, the anaemia may not only be caused by a primary deficiency in folic acid but also by a secondary folic acid deficiency caused by a cobalamin deficiency. Furthermore folic acid deficiency during pregnancy is associated with a risk of miscarriage and embryonal malformation.

In addition a folic acid deficiency can result in the accumulation of metabolites of folic acid metabolism in the organism. For example homocysteine accumulates in the organism when there is a deficiency in folic acid since it cannot be methylated to methionine. Hence folic acid can be regarded as an indicator for the methylation of homocysteine. The methylation of homocysteine to methionine is also reduced in a vitamin B12 deficiency. In both cases there is pathological accumulation of homocysteine in the blood and a homocysteinemia. Homocysteinemia predisposes for various diseases. Thus for example arteriosclerotic cardiovascular diseases, venous thromboses, endothelial damage and an increased stroke risk are for example linked to homocysteinemia. Moreover homocysteinemia is a risk factor for neural tube defects and pre-eclampsia in pregnant women. Hyperhomocysteinemia also encourages disorders of the blood coagulation system and peripheral occlusive arterial disease.

Homocysteine can also be degraded to cysteine by a vitamin B6-dependent metabolic path. Hence adequate levels of vitamin B6 are necessary to maintain a normal homocysteine concentration.

Vitamin B6 is of major importance in protein metabolism. Deficiencies can result in various disturbances of health such as skin changes and disorders of the immune system and nervous system.

Various biochemical parameters are currently used to determine the vitamin B12, vitamin B6 or folic acid status. Vitamin B6 can for example be determined by enzymatic assays or HPLC analyses. Determinations of vitamin B12 and folic acid concentration in serum are also widespread. The concentration of folic acid and vitamin B12 can also be measured in the erythrocytes, in order to investigate a folic acid or vitamin B12 deficiency. However, these measurements are very laborious.

Another method is to determine the blood picture or perform a marrow smear. However, since megaloblastic anaemia occurs with a cobalamin as well as with a folic acid deficiency, no distinction can be made between the blood picture and marrow smear in the case of a folic acid deficiency or vitamin B12 deficiency.

The so-called Schilling test is described in the prior art to detect a disorder of vitamin B12 absorption and the resulting vitamin B12 deficiency. This is a vitamin B12 absorption test in which the excretion of orally administered, radioactively labelled vitamin B12 is determined in the urine. However, this test procedure requires the use of radioactivity, the test is very laborious and results are often unreliable.

Another biochemical parameter that can be used to determine a vitamin B12 deficiency is the concentration of methylmalonic acid (MMA) which increases in the serum and in the urine when there is a deficiency of vitamin B12. The MMA concentration is often already increased in the early stages of a vitamin B12 deficiency, however, the concentration of MMA not only correlates with a vitamin B12 deficiency but it can also have other causes.

An increase in the homocysteine concentration in serum is another indicator for vitamin B12, vitamin B6 and/or folic acid deficiency. However, since homocysteine accumulates in the organism and hence there is an increase in the serum concentration in the case of a vitamin B12 deficiency as well as in the case of a folic acid deficiency, this parameter alone is not sufficient to specifically determine the deficiency which is present.

The close physiological link between folic acid and vitamin B12 and vitamin B6 in metabolism and the resulting similarity of the symptoms in a state of deficiency, make an unequivocal clear diagnosis very difficult. The known tests in the prior art for determining the vitamin B12 and folic acid status often give unreliable results and are usually very complicated to perform.

### SUMMARY OF THE INVENTION

For this reason the object of the present invention was to provide a simple method which enables a reliable assessment of the vitamin status of vitamin B12, vitamin B6 or/and folic acid, and in particular of the intracellular state and additionally enables a differentiation to be made between a vitamin B12 deficiency and a folic acid deficiency.

This object is achieved according to the invention by a method for detecting vitamin B12, vitamin B6 or/and folic acid disorders comprising the determination of holotranscobalamin II (holo TC II), homocysteine (tHCY), methylmalonic acid (MMA), and optionally cystathionine (CSY).

Hence the invention concerns the differential diagnosis of vitamin B12, vitamin B6 or/and folic acid disorders by means of three or four independent parameters. Thus the method according to the invention allows an assessment of the overall state of the vitamin supply of an organism with regard to the vitamins B12, B6 and folic acid.

### BRIEF DESCRIPTION OF THE DRAWINGS

FIG. 1 shows a scheme for the differential diagnosis and monitoring of vitamin B12, B6 or/and folic acid deficiency.

FIGS. 2 to 8 show a schematic representation of the remethylation and transsulphuration of homocysteine in normal metabolism without vitamin B6, B12 or/and folic acid deficiency states and with various vitamin B6, B12 or/and folic acid deficiencies.

FIG. 2 shows a scheme of the remethylation and transsulphuration of homocysteine in the case of a normal metabolism.

FIG. 3 shows a scheme of the remethylation and transsulphuration of homocysteine in the case of a vitamin B12 deficiency.

FIG. 4 shows a scheme of the remethylation and transsulphuration of homocysteine in the case of a folate deficiency.

FIG. 5 shows a scheme of the remethylation and transsulphuration of homocysteine in the case of a vitamin B12 and folate deficiency.

FIG. 6 shows a scheme of the remethylation and transsulphuration of homocysteine in the case of a vitamin B6 deficiency.

FIG. 7 shows a scheme of the remethylation and transsulphuration of homocysteine in the case of a vitamin B12 and vitamin B6 deficiency.

FIG. 8 shows a scheme of the remethylation and transsulphuration of homocysteine in the case of a vitamin B6 and folate deficiency.

FIG. 9 is a scheme of various possible combinations of tests for vitamin deficiency diseases with other tests such as a test for functional iron disorders. In the fields vitamin deficiency/chronic diseases the fields A to E represent a vitamin B12 and folic acid deficiency, the fields C and F represent no vitamin deficiency, the field B represents a vitamin B12 deficiency and the field D represents a vitamin B6 deficiency. Folic acid can be calculated from the difference between field A and field B.

### DESCRIPTION OF THE INVENTION

The measurement parameters holotranscobalamin II, homocysteine, methylmalonic acid and optionally cystathionine are preferably determined from one sample. This may be a sample from a patient. The three or four measurement parameters can be determined in the same or different body fluids, for example blood, blood fractions or urine. The determination is preferably carried out in serum.

The term “vitamin B12” is used herein as a synonymous name for cobalamins and includes all cobalamins that have a biological effect in humans such as methylcobalamin or 5′-deoxyadenosyl cobalamin.

The term “folic acid” is used herein as a collective term for naturally occurring or synthetic compounds which comprise a pteridine ring, p-aminobenzoic acid and one or more glutamic acid residues. The term “folic acid” as used herein also encompasses biologically active forms of these compounds such as tetrahydrofolic acid.

It was surprisingly found according to the invention that rapid and reliable information can be obtained on the vitamin B12, vitamin B6 or/and folic acid status of patients by combining three or four independent parameters. The intracellular vitamin B12, vitamin B6 or/and folic acid status is preferably determined.

In the prior art vitamin B12, B6 and folic acid deficiency is usually determined in serum. However, the informative value of vitamin B12 concentrations in serum is limited due to the lack of sensitivity and specificity. Normal serum values do not always indicate a good vitamin B12 supply and conversely low vitamin B12 concentrations in serum do not always indicate a vitamin B12 deficiency.

The folic acid concentration in plasma only indicates the momentary folic acid balance at the time of blood collection. The folic acid concentration in plasma does not reflect the state of the folic acid stores in the tissues, but is subject to large variations due to the daily uptake of folic acid and changes in folic acid metabolism over time.

Neither the serum concentration of vitamin B12 nor the serum concentration of folic acid give reliable information about the intracellular functional status of these vitamins. The status of vitamin B12 or folic acid in the cell which is essential for an assessment of a deficiency state does not necessarily correlate with the corresponding serum concentrations. The method according to the invention now advantageously allows a determination of the intracellular vitamin B12, vitamin B6 and/or folic acid state.

According to the present invention a determination of the serum concentration of vitamin B12, vitamin B6 and folic acid is not necessary to determine the vitamin B12, vitamin B6 and/or folic acid status but it may be carried out optionally. For this purpose the serum concentrations are additionally determined and used as control values.

The method according to the invention allows an early diagnosis of a vitamin B6, folic acid or/and vitamin B12 deficiency when for example the serum concentrations or the blood picture do not yet indicate a state of deficiency. Such an early diagnosis of a vitamin B12 deficiency can for example be of major importance because a vitamin B12 deficiency can cause neuropsychiatric diseases since the vitamin B12 stores in the brain are very small and are rapidly depleted. Neuropsychiatric diseases can be reversed by a vitamin B12 supplementation if an early diagnosis is made in which case administration of about 1000 μg vitamin B12/day is conceivable.

The method according to the invention allows a classification of vitamin B12, vitamin B6 or/and folic acid states, in particular of vitamin B12, vitamin B6 or/and folic acid deficiencies. By combining the four independent parameters holotrans-cobalamin II, homocysteine, methylmalonic acid and optionally cystathionine, the method according to the invention allows a routine differentiation between a normal vitamin B12, vitamin B6 or folic acid status and a vitamin B12, vitamin B6 or/and folic acid deficiency.

This differentiation is of major importance in order to avoid false treatment. If, for example, a vitamin B12 deficiency is treated with folic acid supplementation, the blood picture becomes normal but the vitamin B12 deficiency still remains which is why there is a remaining risk of secondary diseases such as irreversible nerve degeneration.

In a preferred embodiment the vitamin B12 and folic acid status determined by the method according to the invention is classified into one of the following groups:

(a) vitamin B12, B6 and folic acid deficiency

(b) vitamin B12 and B6 deficiency

(c) folic acid deficiency

(d) no deficiency and optionally

(e) vitamin B6 and folic acid deficiency

(f) vitamin B6 deficiency or

(g) no vitamin B6 deficiency.

The classification is carried out by determining the parameters holotranscobalamin II, homocysteine, methylmalonic acid and optionally cystathionine.

Suitable reference values for the parameter homocysteine (tHCY) are for example in the range of about 3 to 18 μmol/l, preferably about 5 to 15 μmol/l, preferably <about 15 μmol/l, particularly preferably <about 12 μmol/l and especially about 10 μmol/l. The parameter tHCY gives an indication of the homocysteine concentration in the serum. Any value within the reference range such as 12, 13, 14, 15, 16 or 17 μmol/l can be used as a limit for the measurement. About 15 μmol/l is preferably used as the limit.

The reference values for the parameter holotranscobalamin (holo TCII) are preferably in the range of about 20 to 170 pmol/l, preferably about 30 to 160 pmol/l, particularly preferably >about 50 pmol/l, especially >about 30 pmol/l. The parameter holo TC II gives an indication of the homocysteine concentration in the cells. Any value within the reference range such as 28, 29, 30, 31 or 32 pmol/l can be used as a limit for the measurement. About 30 pmol/l is preferably used as the limit.

The reference values for the parameter methylmalonic acid (MMA) are in the range of about 60 to 280 mmol/l, preferably about 70 to 270 mmol/l and in particular <about 270 mmol/l. The parameter MMA is an indicator for B6 and B12 concentrations. Any value within the reference range such as 250, 260, 265, 270, 275 or 280 mmol/l can be used as a limit for the measurement. About 270 mmol/l is preferably used as the limit.

The reference values for the parameter cystathionine are in the range of about 60 to 310 nmol/l, preferably about 65 to 300 nmol/l and in particular <about 300 mmol/l. The parameter cystathionine gives an indication of the B6 concentration. Any value within the reference range such as 280, 290, 295, 300, 305 or 310 mmol/l can be used as a limit for the measurement. About 300 nmol/l is preferably used as the limit.

Thus for example a subdivision like the one shown in the following table is used for a classification into a vitamin B12, vitamin B6 and/or folic acid deficiency.

TABLE

| | Holo Tc II | Homocysteine | Methylmalonic acid | Cystathionine | |
|---|---|---|---|---|---|
| | [pmol/l] | [μmol/l] | [mmol/l] | [nmol/l] | Comment |
| (a)/A | <30 | >15 | <270 | | vitamin B12, B6, folic acid deficiency |
| (b)/B | <30 | >15 | <270 | | vitamin B12, B6 deficiency |
| (c)/A-B | <30 | >15 | <270 | >300 | folic acid deficiency |
| (d)/C/F | >30 | <15 | <270 | <300 | no deficiency |
| (e)/ | <30 | >15 | <270 | <300 | vitamin B6, folic acid deficiency |
| (f)/D | <30 | >15 | <270 | <300 | vitamin B6 deficiency |
| (g)/E | <30 | >15 | <270 | >300 | no vitamin B6 deficiency |

Formula:
A − B = (folic acid + B6 + B12) − (B6 + B12) = folic acid
B − D = (B6 + B12) − B6 = B12

The determined parameters can preferably be evaluated with the aid of a computer for example by means of a suitable software. Furthermore the determined measured values can preferably be shown graphically in the form of diagrams to enable an easy allocation of the measuring ranges to a vitamin B12, vitamin B6 or/and folic acid disorder.

In the method according to the invention it is also possible to predetermine in a simple manner the treatment required for the respective patient depending on the vitamin B12, vitamin B6 or/and folic acid disorder that is determined. Thus, for example, a vitamin B12, B6 and folate supplementation is indicated for a classification in group (a), a vitamin B12 and B6 supplementation is indicated for a classification in group (b), a folate supplementation is indicated for a classification in group (c), no treatment is required for a classification in group (d), optionally a vitamin B6 and folate supplementation is indicated for classification in group (e) and a vitamin B6 supplementation is indicated for classification in group (f).

The vitamins can be supplemented by any suitable type of administration preferably by oral administration.

In the case of vitamin B12 deficiency about 0.1 to 3 mg, preferably about 0.1 to 2 mg, more preferably about 0.1 to 1 mg and in particular about 1 mg, for example 0.9 to 1.1 is administered per day. A folate deficiency is for example supplemented with about 0.1 to 1.5 mg, preferably about 0.1 to 1.0 mg and in particular about 0.5 mg, for example 0.4 to 0.6 mg per day. If a vitamin B6 deficiency is diagnosed, vitamin B6 can be administered at a dose of about 1 to 7 mg, preferably about 1 to 5 mg and in particular about 5 mg, for example 4.5 to 5.5 mg.

Of course, it is possible to administer any combination of the vitamins and also any combination of one or more vitamins with other physiologically tolerated substances such as excipients, aromatics and flavourings or other pharmaceutical substances.

In addition to treatment of vitamin B12, vitamin B6 or/and folic acid disorders, the method according to the invention also allows observation or/and monitoring of the course of treatment or success of treatment in order to thus ensure an optimal use of vitamin B12, vitamin B6 and folic acid preparations (e.g. oral or parenteral vitamin B12, vitamin B6 or folic acid preparations) in the individual patients and also to ensure an optimal medication with regard to dosage and duration of administration.

The method according to the invention is suitable for determining vitamin B12, B6 or/and folic acid disorders of a patient. The method can also be used to determine chronic non-inflammatory (degenerative) diseases that are caused by a vitamin B12, B6 or/and folic acid deficiency which are for example characterized by a normal CRP (C-reactive protein) value.

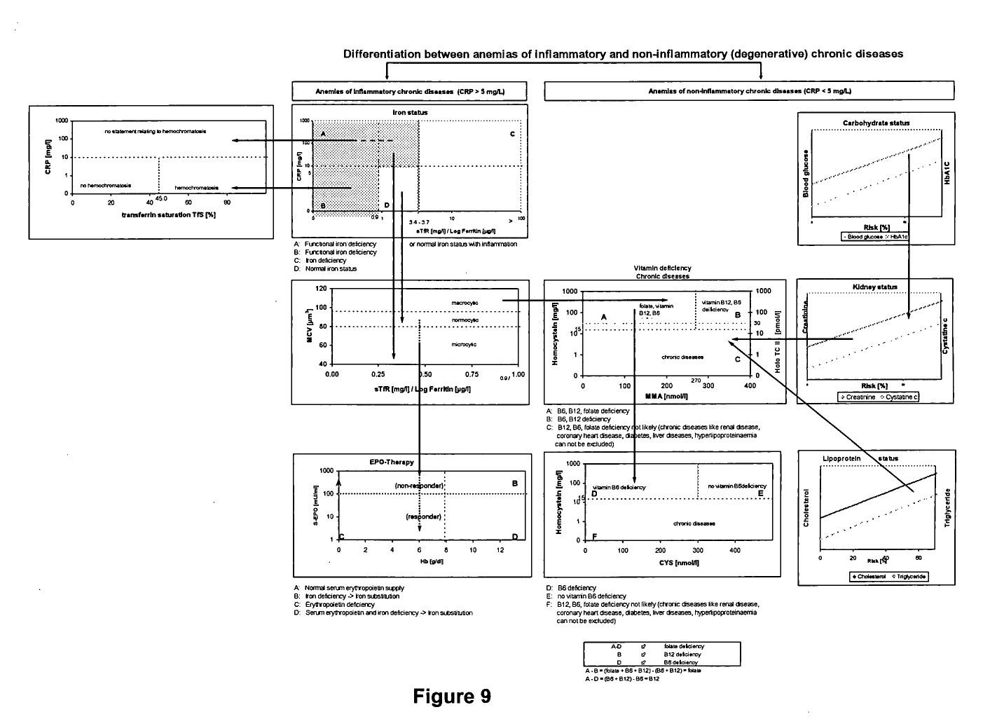

Furthermore the method according to the invention can also be used in combination with other tests, e.g. tests for functional iron disorders (cf. FIG. 9). For example samples from patients with disorders of iron distribution can be subject to the method according to the invention in order to determine a possible vitamin deficiency. In the case of a vitamin deficiency the affected patients then initially receive no erythropoietin but only a vitamin administration.

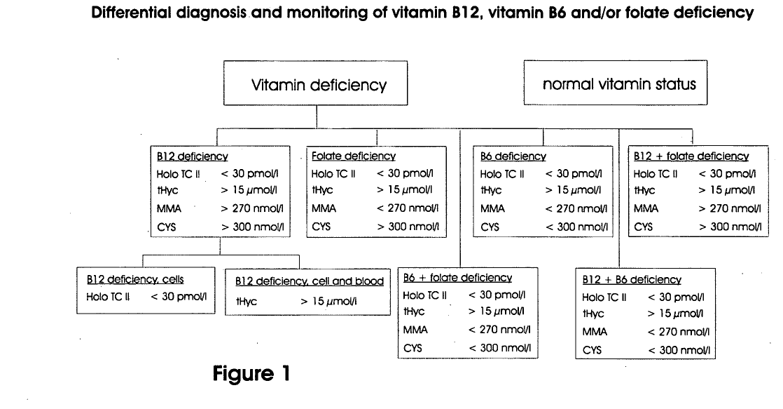

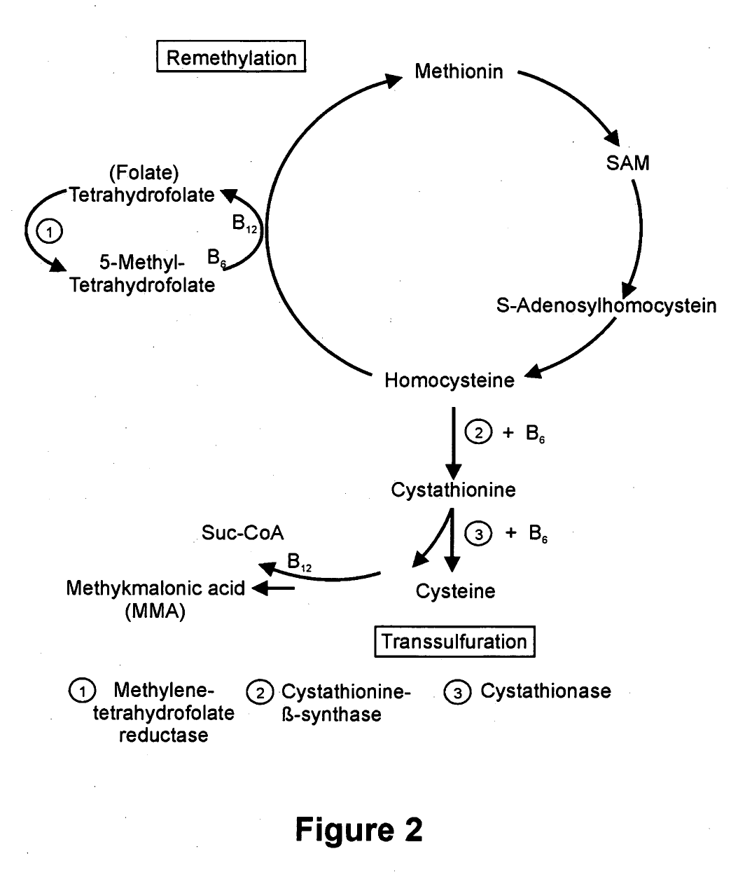

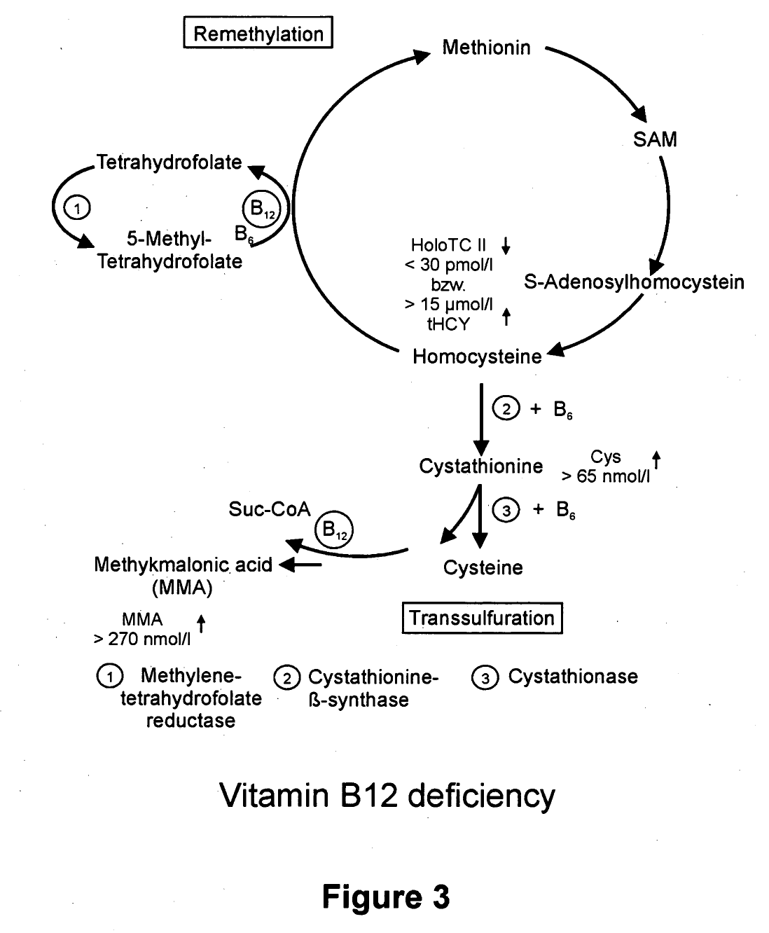

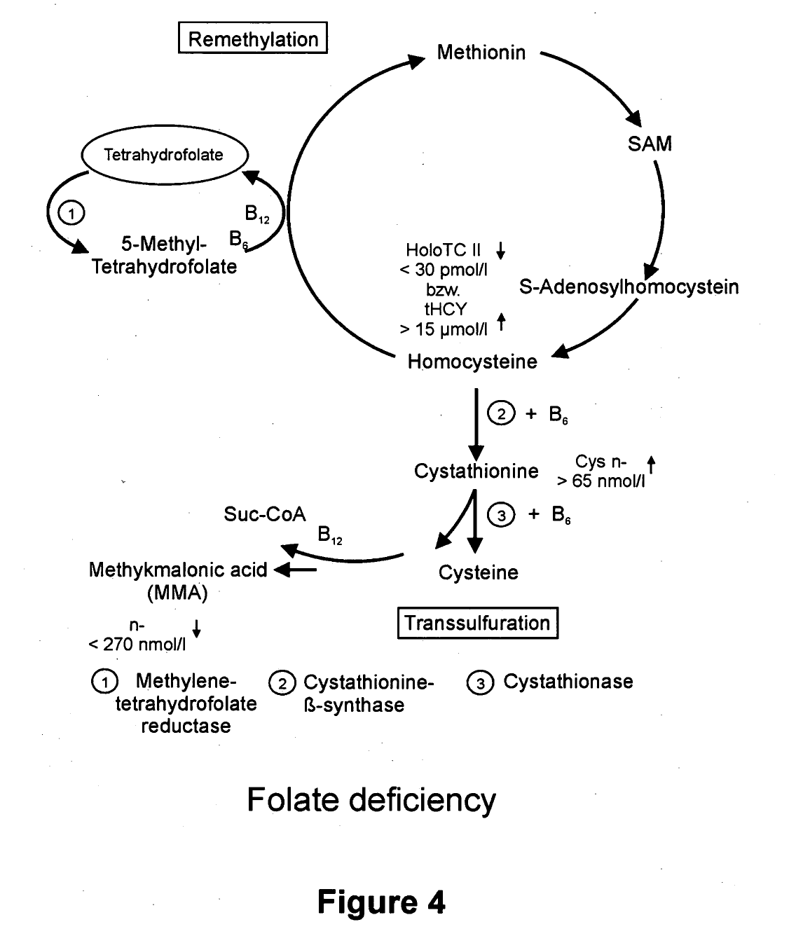

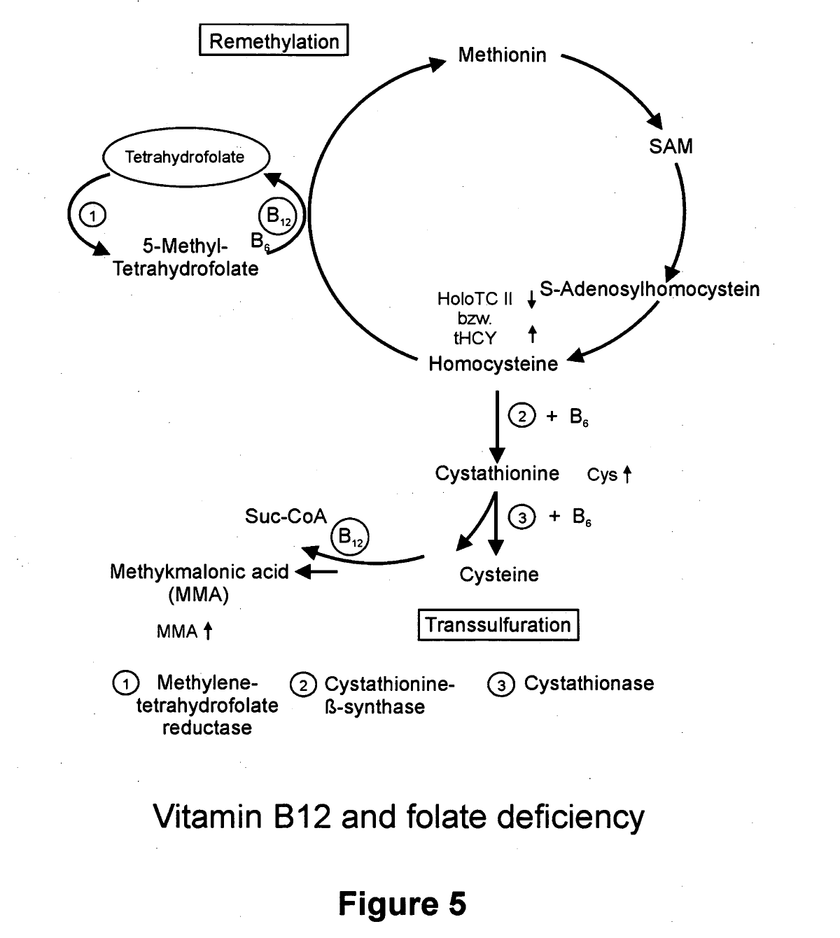

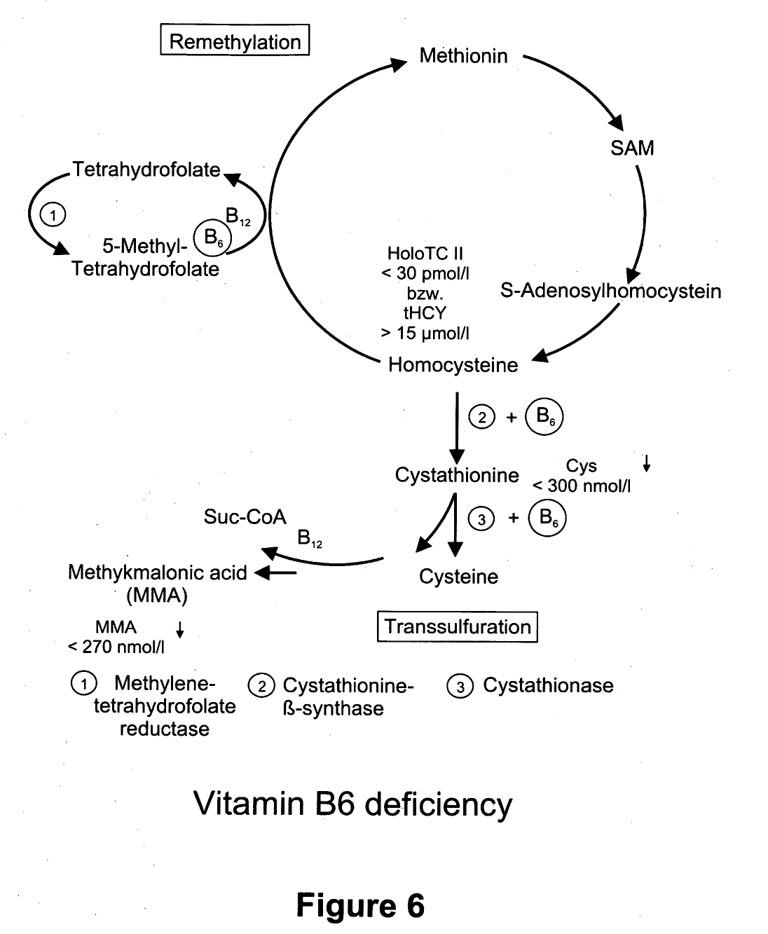

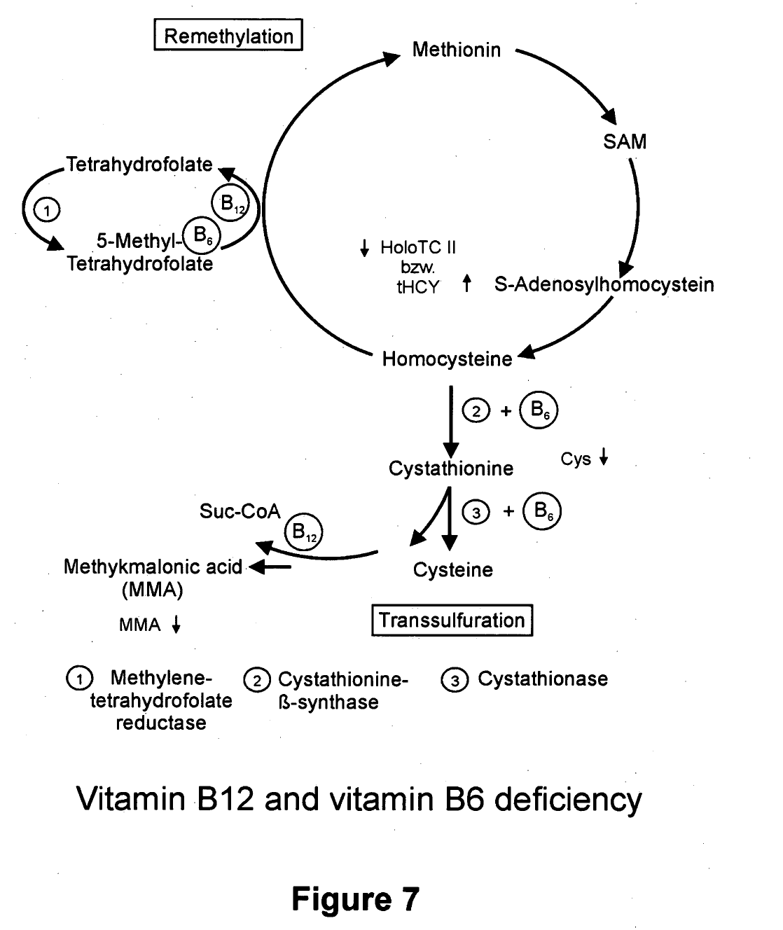

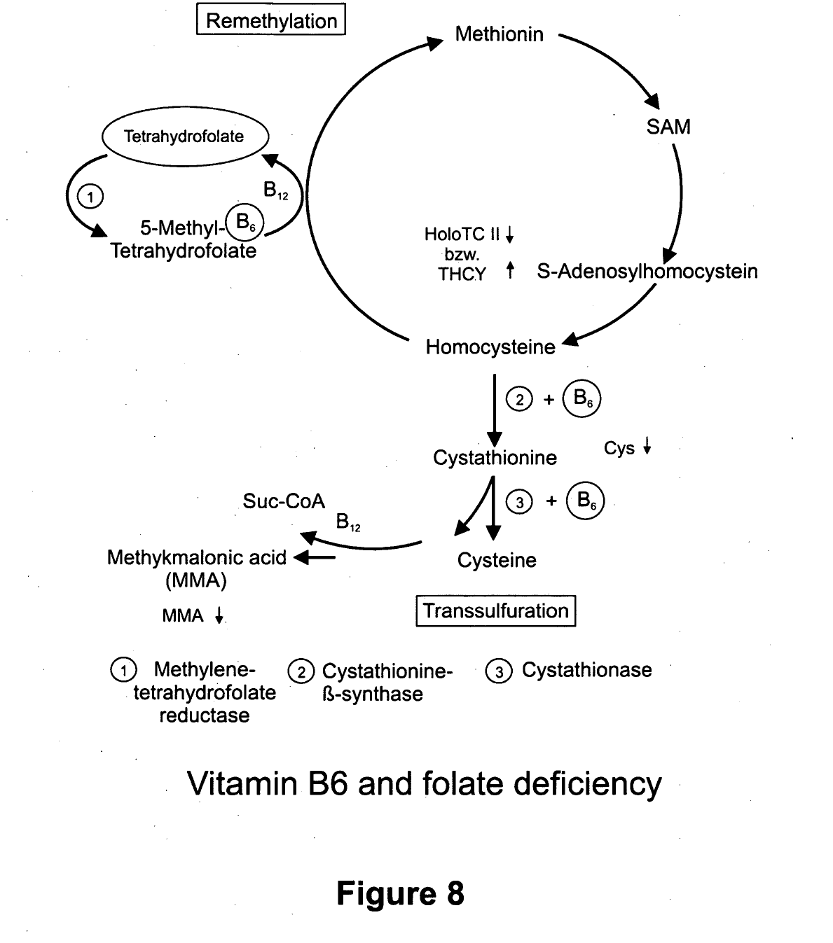

The present invention is further illustrated by FIGS. 1 to 9 and by the example.

In the figures and in the example, the following abbreviations are used:

holo TC II holotranscobalamin II

tHCY total homocysteine

MMA methylmalonic acid

CYS cystathionine

SAM S-adenosylmethionine

Suc-CoA succinyl-Co-A

### EXAMPLE

### Vitamin Substitution in Hyperhomocysteinemia

A patient aged <60 years with non-chronic inflammatory diseases (CRP >15 mg/l) and a hyperhomocysteinemia (tHCY >12 μmol/l) was treated orally for a period of 3 weeks with 1 mg vitamin B12, 1 mg folate and 5 mg vitamin B6. The initial value of t-homocysteine was 13.9 μmol/l. On the 21^{st} day after beginning treatment, the t-homocysteine value was only 8.9 μmol/l, treatment was continued by orally administering the same amount 2× week.

What is claimed is:

1. A method for detecting a vitamin B12 or folic acid disorder comprising:
(a) providing a sample from a patient,
(b) determining the amount of holotranscobalamin II, homocysteine, and methylmalonic acid in the sample, and
(c) relating the determinations from step (b) to a vitamin B12 or folic acid disorder.

2. The method of claim 1 wherein step (b) further includes the determination of cystathionine.

3. The method of claim 1 wherein the sample is serum.

4. The method of claim 1 wherein a differential diagnosis is carried out.

5. The method of claim 1 wherein the disorder is a deficiency of one or more selected from the group consisting of vitamin B12, vitamin B6, and folic acid.

6. The method of claim 5 wherein the deficiency is intracellular.

7. The method of claim 5 further comprising the step of (d) classifying the deficiency into a deficiency selected from the group consisting of:
(a) vitamin B12, vitamin B6, and folic acid deficiency,
(b) vitamin B12 and vitamin B6 deficiency,
(c) folic acid deficiency,
(d) no deficiency,
(e) vitamin B6 and folic acid deficiency,
(f) vitamin B6 deficiency, and
(g) no vitamin B6 deficiency.

8. The method of claim 7 further including the step of (e) recommending a treatment on the basis of the classification resulting in step (d).

9. The method of claim 8 wherein the treatment recommended is selected from the group consisting of a vitamin B12, vitamin B6, and folate supplementation for a classification in group (a); a vitamin B12 and vitamin B6 supplementation for a classification in group (b); a folate supplementation for a classification in group (c); no treatment for a classification in group (d) or (g); a vitamin B6 and folate supplementation for a classification in group (e); and a vitamin B6 supplementation for a classification in group (f).

10. The method of claim 8 further comprising step (f) observing or monitoring the course or success of treatment.
